(19) United States
(12) Patent Application Publication
(10) Pub. No.: US 2020/0072816 A1
(43) Pub. Date: Mar. 5, 2020

(54) GAS SENSOR DEVICE, MANUFACTURING METHOD THEREOF, AND GAS EVALUATION APPARATUS

(71) Applicant: FUJITSU LIMITED, Kawasaki-shi (JP)

(72) Inventors: Satoru Momose, Atsugi (JP); Osamu Tsuboi, Kawasaki (JP); Michio Ushigome, Atsugi (JP)

(73) Assignee: FUJITSU LIMITED, Kawasaki-shi (JP)

(21) Appl. No.: 16/537,497

(22) Filed: Aug. 9, 2019

(30) Foreign Application Priority Data
Aug. 29, 2018 (JP) 2018-160403

(51) Int. Cl. G01N 33/497; G01N 27/12

(52) U.S. Cl. CPC G01N 33/497; G01N 2033/4975; G01N 27/125

(57) ABSTRACT

A gas sensor device includes: a pair of electrodes; a conductor that electrically connects the pair of electrodes to each other; and at least one of a thiolate anion and an organic compound having a mercapto group, which is disposed on a surface of the conductor. A gas evaluation apparatus includes a first gas sensor device as described above and a second gas sensor device which has neither the mercapto group nor the thiolate anion disposed on a surface of its conductor.

### CROSS-REFERENCE TO RELATED APPLICATION

This application is based upon and claims the benefit of priority of the prior Japanese Patent Application No. 2018-160403, filed on Aug. 29, 2018, the entire contents of which are incorporated herein by reference.

### FIELD

The present disclosure relates to a gas sensor device, a manufacturing method thereof, and a gas evaluation apparatus using the gas sensor device.

### BACKGROUND

Halitosis is mainly caused by a gas produced by the activity of bacteria in the oral cavity. Therefore, the halitosis acts not only as a problem on etiquette, but also reflects the physical condition of the oral cavity. Thus, a method in which a dentist uses the sense of smell for diagnosis or measures a causative agent of halitosis with an analysis device of, for example, gas chromatography has been performed.

However, there is a problem in that the determination based on the sense of smell reflects individual differences, and thus it is difficult to ensure quantitativeness. The analysis device of, for example, gas chromatography has a problem in that skill is required in operation and analysis of a result.

Therefore, there is an attempt to evaluate halitosis using a sensing device which is cheaper and has a simpler configuration. For example, a technology of separately measuring an air in the oral cavity and an air exhaled from the lungs by using a metal-oxide semiconductor as a detection element is disclosed (for example, see Japanese Laid-open Patent Publication No. 2004-108861).

As also disclosed in Japanese Laid-open Patent Publication No. 2004-108861, main generation sources of hydrogen sulfide and mercaptans contained in halitosis are different from each other. Thus, if the hydrogen sulfide and the mercaptans are separately measured, it is possible to easily obtain information required for measures against halitosis.

However, as in Japanese Laid-open Patent Publication No. 2004-108861, in a case where a metal-oxide semiconductor is used as a detection element, it is difficult to separately measure hydrogen sulfide and mercaptans because a reaction with hydrogen sulfide is similar to a reaction with mercaptans (alias thiols).

Thus, a gas sensor device capable of selectively measuring hydrogen sulfide with distinguishing hydrogen sulfide from mercaptans is required.

An object of the technology in the disclosure is to provide a gas sensor device and a manufacturing method thereof capable of selectively measuring hydrogen sulfide among hydrogen sulfide and mercaptans, and a gas evaluation apparatus using the gas sensor device.

### SUMMARY

According to an aspect of the embodiments, a gas sensor device includes: a pair of electrodes; a conductor that electrically connects the pair of electrodes to each other; and at least one of a thiolate anion and an organic compound having a mercapto group, which is disposed on a surface of the conductor. And a gas evaluation apparatus includes a first gas sensor device as described above and a second gas sensor device which has neither the mercapto group nor the thiolate anion disposed on a surface of its conductor.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1A is a top view schematically illustrating an example of a gas sensor device according to the disclosure;

FIG. 1B is a sectional view schematically illustrating the gas sensor device in FIG. 1A;

FIG. 2A is a sectional view schematically illustrating an example of a producing method of the example of the gas sensor device in the disclosure (part 1);

FIG. 2B is a sectional view schematically illustrating the example of the producing method of the example of the gas sensor device in the disclosure (part 2);

FIG. 2C is a sectional view schematically illustrating the example of the producing method of the example of the gas sensor device in the disclosure (part 3);

FIG. 2D is a sectional view schematically illustrating the example of the producing method of the example of the gas sensor device in the disclosure (part 4);

FIG. 3 is a sectional view schematically illustrating a form in which an organic compound having a mercapto group is disposed on a surface of a conductor;

FIG. 4 is a configuration diagram illustrating the example of the gas evaluation apparatus in the disclosure;

FIG. 5 is a flowchart illustrating an example of gas measurement using the gas evaluation apparatus in the disclosure;

FIG. 6 is a configuration diagram illustrating another example of the gas evaluation apparatus in the disclosure;

FIG. 7 is a flowchart illustrating an example of an odor evaluation method using the gas evaluation apparatus in the disclosure;

FIG. 8 illustrates a response profile of a resistance value to hydrogen sulfide in a gas sensor device in Example 1;

FIG. 9 illustrates a response profile of the resistance value to methyl mercaptan in the gas sensor device in Example 1;

FIG. 10 illustrates a response profile of the resistance value to hydrogen sulfide in a gas sensor device in Comparative Example 1; and

FIG. 11 illustrates a response profile of the resistance value to methyl mercaptan in the gas sensor device in Comparative Example 1.

### DESCRIPTION OF EMBODIMENTS

(Gas Sensor Device)

A gas sensor device according to the disclosure includes at least a pair of electrodes and a conductor, and, if required, further includes other members. In the gas sensor device, at least any of an organic compound having a mercapto group and a thiolate anion is disposed on the surface of the conductor.

As also disclosed in Japanese Laid-open Patent Publication No. 2004-108861, main generation sources of hydrogen sulfide and mercaptans contained in halitosis are different from each other. Thus, if the hydrogen sulfide and the mercaptans are separately measured, it is possible to easily obtain information required for measuring halitosis.

For example, the known technology 1 (Journal of Dentistry vol. 35 (2007), pp. 552-557) discloses that halitosis becomes noticeable if the total concentration of volatile sulfide (mainly, hydrogen sulfide and methyl mercaptan) contained in an air in the mouth is more than 1 ppm. The known technology 2 (Journal of Medical Microbiology (2005), vol. 54, pp. 889-895) discloses that the concentration of hydrogen sulfide in an air in the mouth is high in a case where the tongue has become largely furred. The known technology 3 (Journal of Periodontology (1996), vol. 63, No. 9, pp. 783-789) discloses that the main component causing halitosis is hydrogen sulfide and methyl mercaptan and that a ratio of the concentration of methyl mercaptans to the concentration of hydrogen sulfide in an air in the mouth increases and becomes more than 1 in a case where periodontal disease is in progress.

Thus, the inventors performed a close examination for providing a gas sensor device capable of selectively measuring hydrogen sulfide in order to separately measure hydrogen sulfide and mercaptans. As a result, the inventors found that at least any of a thiolate anion and an organic compound having a mercapto group was disposed on the surface of a conductor across a pair of electrodes, and thereby it was possible to selectively measure hydrogen sulfide among hydrogen sulfide and mercaptans, and finished the technology in the disclosure.

<Pair of Electrodes>

The pair of electrodes in the gas sensor device is not particularly limited and may be appropriately selected in accordance with the purpose. The material of the pair of electrodes is not particularly limited and may be appropriately selected in accordance with the purpose. Examples of the material thereof include gold (Au) and platinum (Pt).

The shape of the pair of electrodes is not particularly limited and may be appropriately selected in accordance with the purpose. Examples of the shape thereof include a flat-plate shape. The size of the pair of electrodes is not particularly limited and may be appropriately selected in accordance with the purpose. Examples of the size thereof include a size which is equal to or less than a square of 1 cm. The structure of the pair of electrodes is not particularly limited and may be appropriately selected in accordance with the purpose. Regarding each of the pair of electrodes, the material, the size, and the structure may be the same as or different from each other.

A method of forming the pair of electrodes is not particularly limited and may be appropriately selected in accordance with the purpose. For example, a physical vapor deposition method is exemplified.

<Conductor>

The conductor electrically connects the pair of electrodes. At least any of a thiolate anion and an organic compound having a mercapto group is disposed on the surface of the conductor. The thiolate anion is an anion which may be obtained by removing hydrogen in a mercapto group of an organic compound (also referred to as thiol) having the mercapto group.

The term of “at least any of a thiolate anion and an organic compound having a mercapto group” is referred to as “an S-containing organic compound” below. S refers to an element symbol of sulfur. Since the S-containing organic compound is disposed on the surface of the conductor, hydrogen sulfide among the hydrogen sulfide and mercaptans is selectively combined to the conductor. As a result, the gas sensor device is capable of selectively measuring hydrogen sulfide among the hydrogen sulfide and mercaptans.

Preferably, in the gas sensor device, the material of the conductor is gold, and at least the organic compound is disposed in an area which is on the surface of the conductor and in which a crystal plane of the gold is (111).

Descriptions that gold as an example is used for the conductor, and hydrogen sulfide may be selectively measured rather than mercaptans in the gas sensor device will be made below.

Hydrogen sulfide and mercaptans are capable of reversibly forming bonds with the conductor (for example, gold) at room temperature. If thiol (organic compound having 10 or more carbon atoms and a mercapto group (—SH)) having no volatility at ambient temperature is brought into contact with the surface of a gold thin film, the thiol is preferentially combined to a portion of the surface of the gold thin film. At this portion, the mercapto group is easily combined (representatively, a portion at which the (111) oriented surface is exposed). The bond is reversible, however, since thiol combined with gold is non-volatile, even in a case where the combination is temporarily released, a thiol molecule stays in the vicinity thereof, and thus recombination may easily occur. Therefore, coating of the surface of the gold thin film by thiol does not easily change. In a case where gaseous mercaptans (thiol) come into contact with the surface of the gold thin film, of which thiol is combined at the portion at which the mercapto group is easily combined, the entirety of the portion at which the mercapto group is easily combined is fully occupied. Thus, it is difficult to form a new bond. Hydrogen sulfide is more reactive to gold than mercaptans. Thus, a bond may be formed even at a portion at which combination of mercaptan has difficulty. That is, hydrogen sulfide among the hydrogen sulfide and mercaptans is selectively combined to the surface of the gold thin film.

If the hydrogen sulfide is combined to the surface of the gold thin film, metallicity is lost at this portion. Thus, the cross-sectional area as the conductor decreases, and an electrical resistance value increases. As a result of such a phenomenon, in the technology of the disclosure, the gas sensor device selectively reacts with hydrogen sulfide and is capable of selectively measuring hydrogen sulfide among the hydrogen sulfide and mercaptans.

Accordingly, a gas evaluation apparatus capable of selectively measuring hydrogen sulfide among the hydrogen sulfide and mercaptans may be constituted by the gas sensor device in the disclosure.

The gas sensor device configured by a gold thin film having a surface on which thiol is not disposed reacts with both hydrogen sulfide and mercaptans. Therefore, it is possible to obtain a signal corresponding to the concentration of mercaptans from a signal of a difference between a resistance change of the gas sensor device having such a configuration and a resistance change of the gas sensor device in the technology in the disclosure.

Thus, a gas evaluation apparatus capable of measuring the concentrations of both hydrogen sulfide and mercaptans may be configured by using both the gas sensor devices. The material of the conductor is not particularly limited so long as the material is a conductor, and may be appropriately selected in accordance with the purpose. Gold and platinum-group metal are preferable. Examples of the platinum-group metal include platinum, palladium, ruthenium, rhodium, and iridium.

Hydrogen sulfide and mercaptans are capable of forming coordination bonds with various metal atoms and various metal ions. However, other gas species have coordination ability with metal atoms and metal ions. Thus, normally, formation of the coordination bond is performed competitively between these gas species. Accordingly, it is possible to produce a sensor device that more selectively reacts with most types of gases by using a material which is inert to the gases and is capable of forming a reversible bond to hydrogen sulfide and mercaptans. From this viewpoint, gold and platinum-group metal are preferable as the material of the conductor.

In a case where a gas sensor that performs measurement using a resistance change of a semiconductor material such as an organic semiconductor is constructed, the sensor generally exhibits some responses to a gas component having polarity. Thus, it is inevitable that a response to water molecules which are a very large number of polar molecules in an air, that is, to humidity, occurs. For example, in a case where an air in an oral cavity having very high humidity is set as a measurement target, it is desirable that the measurement result is not influenced by the humidity. From this viewpoint, gold and platinum-group metal are preferable as the material of the conductor.

The organic compound having a mercapto group is not particularly limited and may be appropriately selected in accordance with the purpose. Thiol having one mercapto group, thiol having two mercapto groups, and thiol having three or more mercapto groups are exemplified.

Preferably, the organic compound having a mercapto group does not have volatility at ambient temperature. From this viewpoint, the number of carbon atoms in the organic compound having a mercapto group is equal to or more than 5. The upper value of the carbon atoms is not particularly limited and may be appropriately selected in accordance with the purpose. For example, an organic compound having 20 or less carbon atoms is exemplified. Examples of the organic compound having a mercapto group include alkyl mercaptan having 10 to 20 carbon atoms. Examples of the organic compound having a mercapto group include non-aromatic dithiols having 5 to 10 carbon atoms. Examples of the dithiol include 1,5-pentanedithiol, 1,6-hexanedithiol, 1,8-octanedithiol, 1,9-nonanedithiol, and 2,2′-ethylene dioxydiethanethiol. The materials may be used singly or may be used in combination of two kinds or more thereof.

In a case where the thiolate anion is derived from thiol having two or more mercapto groups, the thiolate anion may be obtained in a manner that hydrogen in all mercapto groups of the thiol is not removed. For example, the thiolate anion may be generated by removing one hydrogen atom from the thiol or may be generated by removing two or more hydrogen atoms from the thiol.

The amount of the S-containing organic compound on the surface of the conductor is not particularly limited and may be appropriately selected in accordance with the purpose.

A method of disposing the S-containing organic compound on the surface of the conductor is not particularly limited and may be appropriately selected in accordance with the purpose. For example, a method of immersing the conductor in a solution which contains the S-containing organic compound is exemplified. The concentration of the S-containing organic compound in the solution is not particularly limited and may be appropriately selected in accordance with the purpose.

The shape of the conductor is not particularly limited and may be appropriately selected in accordance with the purpose. Examples of the shape thereof include a film shape. An average thickness of the film-like conductor is not particularly limited and may be appropriately selected in accordance with the purpose. For example, a thickness of 1 nm to 50 nm is exemplified. The size of the conductor is not particularly limited and may be appropriately selected in accordance with the purpose. Examples of the size thereof include a size in which the length between the pair of electrodes is 100 μm to 5 mm, and the width is 10 μm to 500 μm. The structure of the conductor is not particularly limited and may be appropriately selected in accordance with the purpose.

The S-containing organic compound may be disposed on a side surface of a conductive film as the conductor or may be disposed on both surfaces thereof.

An Interaction between the S-containing organic compound and the conductor when the S-containing organic compound is disposed on the surface of the conductor is not particularly limited and may be appropriately selected in accordance with the purpose. The interaction may cause a coordinate bond or a covalent bond. For example, metal (M) contained in the conductor and sulfur (S) in the mercapto group may be combined with each other between the conductor and the organic compound having the mercapto group, and thus a bond of M-S may be formed. In this case, hydrogen (H) in the mercapto group may be removed from the organic compound.

(Manufacturing Method of Gas Sensor Device)

In the disclosure, a manufacturing method of the gas sensor device includes at least a contact process and further includes other processes such as a washing process, if required. The manufacturing method of the gas sensor device is a preferred manufacturing method of the gas sensor device in the disclosure.

<Contact Process>

The contact process is a process of bringing a structural body including a pair of electrodes and a conductor that electrically connects the pair of electrodes to each other into contact with a solution containing an organic compound having a mercapto group such that the conductor comes into contact with the solution.

<<Structural Body>>

The structural body includes a pair of electrodes and a conductor that electrically connects the pair of electrodes to each other.

<<<Pair of Electrodes>>>

Examples of the pair of electrodes include the pair of electrodes exemplified in the descriptions of the gas sensor device in the disclosure.

<<<Conductor>>>

Examples of the conductor include the conductor which is exemplified in the descriptions of the gas sensor device in the disclosure and is a conductor before at least any of a thiolate anion and an organic compound having a mercapto group is disposed on the surface of the conductor.

<<<Organic Compound Having Mercapto Group>>>

Examples of the organic compound having a mercapto group include the organic compound which has the mercapto group and is exemplified in the descriptions of the gas sensor device in the disclosure.

<<<Solution>>>

A solvent in the solution is not particularly limited and may be appropriately selected in accordance with the purpose. For example, alcohol is exemplified. Examples of the alcohol include methanol. The concentration of the organic compound having the mercapto group in the solution is not particularly limited and may be appropriately selected in accordance with the purpose. For example, a concentration of 0.1 volume % to 5 volume % is exemplified. The temperature of the solution is not particularly limited and may be appropriately selected in accordance with the purpose. For example, the ambient temperature is exemplified.

A method of bringing the conductor into contact with the solution in the contact process is not particularly limited and may be appropriately selected in accordance with the purpose. For example, a method of immersing the structural body in the solution and a method of coating the conductor with the solution are exemplified. In the contact process, the time to bring the conductor into contact with the solution is not particularly limited and may be appropriately selected in accordance with the purpose. For example, time of 10 seconds to 10 minutes is exemplified.

<Washing Process>

After the contact process, preferably, a washing process using a washing liquid is performed in order to remove the excessive organic compound having a mercapto group on the surface of the conductor. The washing process is performed, for example, by immersing the structural body in the washing liquid. Examples of the washing liquid include alcohol. Examples of the alcohol include methanol. The washing liquid does not include the organic compound having a mercapto group.

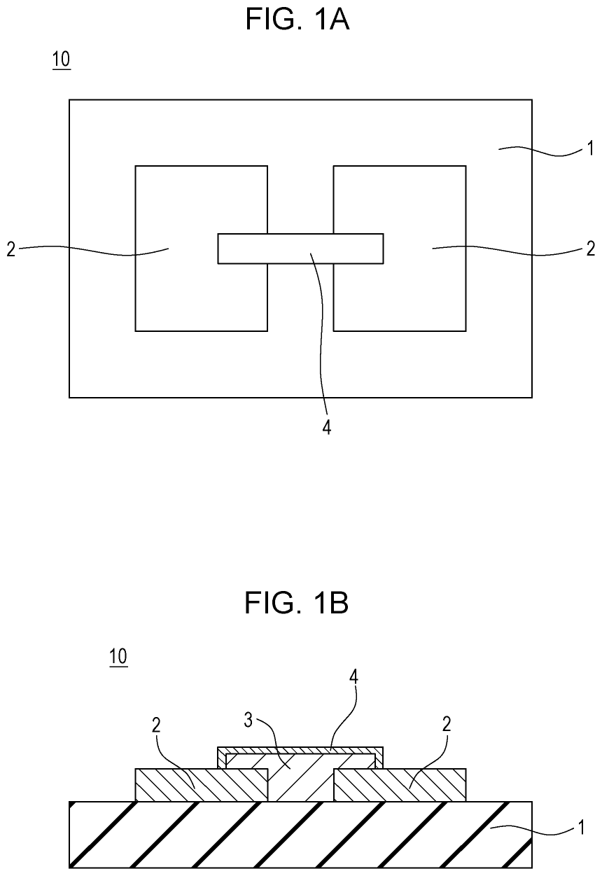

Here, an example of the gas sensor device in the disclosure will be described with reference to the drawings. FIG. 1A is a top view schematically illustrating an example of a gas sensor device according to the disclosure.

FIG. 1B is a sectional view schematically illustrating the gas sensor device in FIG. 1A. A gas sensor device 10 illustrated in FIGS. 1A and 1B includes an insulating substrate 1, a pair of electrodes 2, a conductor 3, and an organic compound 4 having a mercapto group.

In the gas sensor device 10, the conductor 3 is formed to straddle the pair of electrodes 2 formed on a substrate 1. The organic compound 4 having a mercapto group is disposed on the surface of the conductor 3.

For example, the gas sensor device 10 may be manufactured by a method as follows.

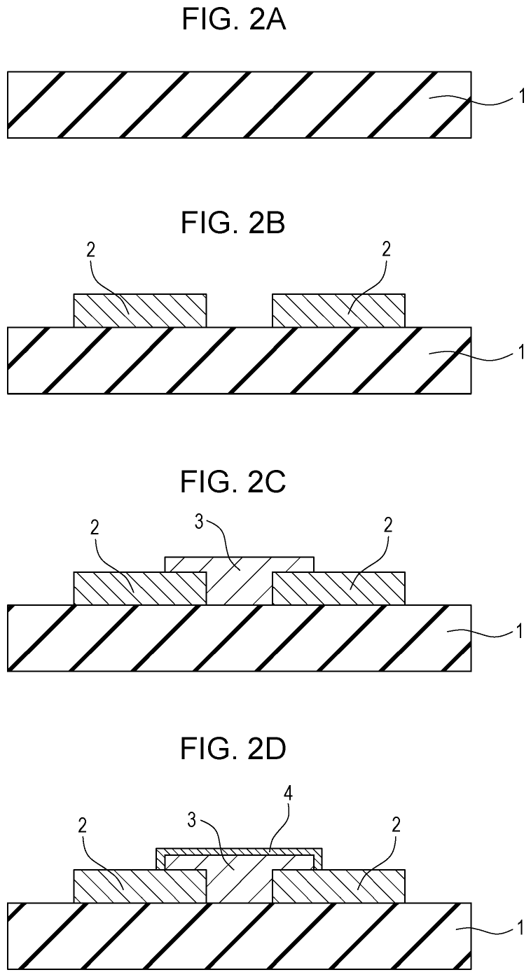

Firstly, the substrate 1 is prepared (FIG. 2A).

Then, the pair of electrodes 2 is formed on the substrate 1 (FIG. 28). The pair of electrodes 2 may be formed by a vacuum evaporation method, for example.

Then, the conductor 3 is formed to straddle the pair of electrodes 2 (FIG. 2C). The conductor 3 may be formed by a vacuum evaporation method, for example.

Then, the organic compound 4 having a mercapto group is formed on the surface of the conductor 3 (FIG. 2D). Examples of a method of disposing the organic compound 4 having a mercapto group on the surface of the conductor 3 include a method of immersing a structural body which is illustrated in FIG. 2C and includes the substrate 1, the pair of electrodes 2, and the conductor 3, in a solution containing the organic compound 4 having a mercapto group. In this manner, the gas sensor device 10 is obtained.

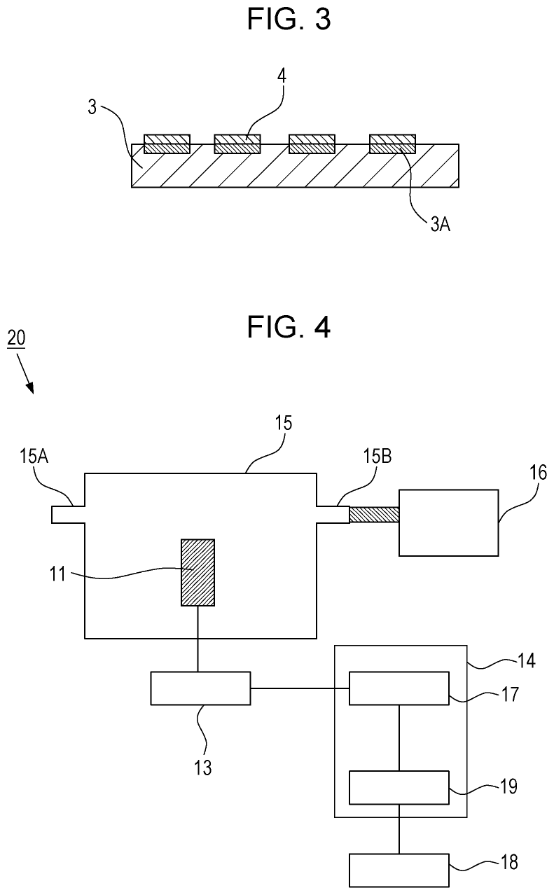

FIGS. 1A, 1B, and 2D illustrate that the organic compound having a mercapto group is disposed in the entirety of the surface of the conductor 3 (that is, the entirety of the surface of the conductor 3 is coated with the organic compound having a mercapto group). However, a form in which the organic compound having a mercapto group is disposed on the surface of the conductor 3 is not limited to a form of coating the entirety of the surface of the conductor. For example, as illustrated in FIG. 3, the organic compound having a mercapto group may be partially disposed on the surface of the conductor 3. In FIG. 3, the organic compound 4 having a mercapto group is disposed on a specific crystal plane 3A ((111) plane) of a gold thin film as the conductor 3.

(Gas Evaluation Apparatus)

A gas evaluation apparatus in the disclosure includes at least a first gas sensor device and a resistance measurement unit. Preferably, the gas evaluation apparatus includes a second gas sensor device and, if required, further includes other members such as a calculation unit and a measurement chamber.

<First Gas Sensor Device>

The first gas sensor device includes a pair of first electrodes and a first conductor that electrically connects the pair of first electrodes to each other. At least any of an organic compound having a mercapto group and a thiolate anion is disposed on the surface of the first conductor. The first gas sensor device is the gas sensor device in the disclosure. That is, the pair of first electrodes in the first gas sensor device is the pair of electrodes in the gas sensor device in the disclosure. A conductor in the first gas sensor device, herein after called as a first conductor, is the conductor in the gas sensor device in the disclosure as disclosed in FIGS. 1A to 3.

<Resistance Measurement Unit>

The resistance measurement unit is not particularly limited so long as the unit is capable of measuring the resistance of the first gas sensor device. The resistance measurement unit may be appropriately selected in accordance with the purpose, and for example, a potentiometer is exemplified. The resistance measurement unit may be capable of further measuring the resistance of the second gas sensor device. For example, a potentiometer is connected to each of the first gas sensor device and the second gas sensor device, and thus the resistance of each gas sensor device is measured. That is, the resistance measurement unit includes a first resistance measuring instrument (for example, potentiometer) connected to the first gas sensor device and a second resistance measuring instrument (for example, potentiometer) connected to the second gas sensor device.

<Second Gas Sensor Device>

The second gas sensor device includes a pair of second electrodes and a second conductor that electrically connects the pair of second electrodes to each other. At least any of an organic compound having a mercapto group and a thiolate anion is not disposed on the surface of the second conductor.

<<Pair of Second Electrodes>>

The pair of second electrodes is not particularly limited and may be appropriately selected in accordance with the purpose. For example, the pair of second electrodes described in the gas sensor device in the disclosure is exemplified.

<<Second Conductor>>

The second conductor is not particularly limited and may be appropriately selected in accordance with the purpose. Examples of the second conductor include the conductor which is exemplified in the descriptions of the gas sensor device in the disclosure before at least any of the organic compound having a mercapto group and a thiolate anion is disposed on the surface of the conductor. The second conductor does not have a mercapto group or thiolate anon disposed thereon.

<Calculation Unit>

The calculation unit is not particularly limited so long as the unit is capable of selectively measuring the concentration of hydrogen sulfide based on the resistance measured by the resistance measurement unit, among hydrogen sulfide and mercaptans. The calculation unit may be appropriately selected in accordance with the purpose and includes a central processing unit (CPU) and a memory, for example.

As another example, the calculation unit is not particularly limited so long as the unit is capable of calculating the concentration of each of hydrogen sulfide and mercaptans based on the resistance measured by the resistance measurement unit. The calculation unit may be appropriately selected in accordance with the purpose and includes a central processing unit (CPU) and a memory, for example.

The calculation unit has a data table indicating a correspondence relation between the resistance, and hydrogen sulfide or mercaptans. The calculation unit calculates the concentration of each of hydrogen sulfide and mercaptans from the resistance measured by the resistance measurement unit, based on the data table.

The calculation unit may evaluate odor based on the concentration of hydrogen sulfide and the concentration of mercaptans, which are obtained from a measurement result of the first gas sensor device and a measurement result of the second gas sensor device.

The calculation unit outputs an evaluation result of odor based on the concentration of hydrogen sulfide and the concentration of mercaptans, which are obtained from the measurement result of the first gas sensor device and the measurement result of the second gas sensor device. The evaluation result is output to the display unit, for example.

For example, the calculation unit evaluates the odor based on (1) the sum value of the concentration of hydrogen sulfide and the concentration of mercaptans and (2) a ratio of the concentration of mercaptans to the concentration of hydrogen sulfide. More specifically, for example, the calculation unit evaluates halitosis in a manner as follows.

The calculation unit measures the concentration of hydrogen sulfide and the concentration of mercaptans in breath exhaled from the mouth. In a case where the sum value of the concentration of hydrogen sulfide and the concentration of mercaptans is less than a threshold value (for example, 1 ppm), the calculation unit outputs an evaluation result indicating that there is no problem. In a case where the sum value of the concentration of hydrogen sulfide and the concentration of mercaptans is equal to or more than the threshold value, the calculation unit further evaluates the ratio of the concentration of mercaptans to the concentration of hydrogen sulfide. In a case where the ratio of the concentration of mercaptans to the concentration of hydrogen sulfide is equal to or less than 1, the calculation unit outputs an evaluation result indicating a possibility that scraps (e.g., food particles) have accumulated in the mouth. In a case where the ratio of the concentration of mercaptans to the concentration of hydrogen sulfide is more than 1, the calculation unit outputs an evaluation result indicating a possibility that periodontal disease is in progress, to the display unit.

In the first gas sensor device, the concentration of hydrogen sulfide among hydrogen sulfide and mercaptans may be selectively measured. Therefore, the gas evaluation apparatus is capable of selectively measuring the concentration of hydrogen sulfide among hydrogen sulfide and mercaptans.

In the second gas sensor device, both hydrogen sulfide and mercaptans may be measured. Therefore, it is possible to measure the concentration of mercaptans from a signal of a difference between a resistance change of the first gas sensor device and a resistance change of the second gas sensor device. That is, it is possible to separately measure the concentrations of both hydrogen sulfide and mercaptans by using the first gas sensor device and the second gas sensor device. For example, it is possible to evaluate halitosis by separately measuring the concentrations of both hydrogen sulfide and mercaptans.

The gas evaluation apparatus in the technology in the disclosure and the gas measurement method using the gas evaluation apparatus will be described with the drawings. A gas evaluation apparatus 20 illustrated in FIG. 4 includes a first gas sensor device 11, a resistance measurement unit 13, a calculation unit 14, a measurement chamber 15, a suction device 16, and a display unit 18. In the gas evaluation apparatus 20, the first gas sensor device 11 is disposed in the measurement chamber 15 including a gas inlet 15A and a gas outlet 15B. The gas outlet 15B is connected to the suction device 16. The first gas sensor device 11 is connected to the resistance measurement unit 13. The resistance measurement unit 13 includes a resistance measuring instrument that measures the resistance change of the first gas sensor device 11, for example. The resistance measurement unit 13 is connected to the calculation unit 14. The calculation unit 14 includes a CPU 17 and a memory 19. The calculation unit 14 is connected to the display unit 18.

In the gas evaluation apparatus illustrated in FIG. 4, while breath exhaled from the mouth is caused to flow from the gas inlet 15A into the measurement chamber 15, the gas in the measurement chamber 15 is sucked by the suction device 16 connected to the gas outlet 15B. In this manner, hydrogen sulfide and mercaptans contained in the breath exhaled from the mouth pass by the surface of the conductor in the first gas sensor device 11 disposed in the measurement chamber 15. The resistance change of the first gas sensor device 11 at this time is measured by the resistance measurement unit 13. The calculation unit 14 calculates the concentration of hydrogen sulfide based on the resistance change measured by the resistance measurement unit 13. The measurement result is output to the display unit 18. The display unit 18 is a monitor, for example.

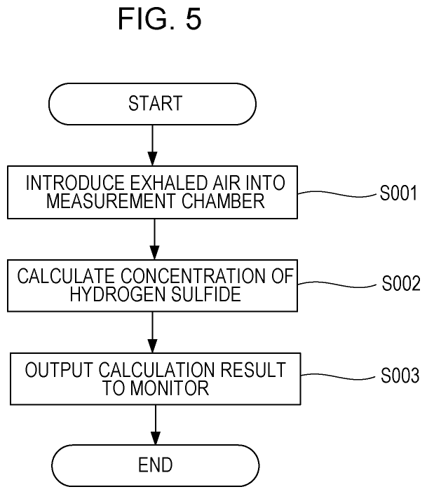

An example of gas evaluation using the gas evaluation apparatus illustrated in FIG. 4 will be described with reference to the flowchart in FIG. 5. Firstly, breath (exhalation) exhaled from the mouth is put into the measurement chamber 15 (S001). Then, the concentration of hydrogen sulfide in the exhalation is calculated by the first gas sensor device 11, the resistance measurement unit 13, and the calculation unit 14 (S002). Then, the calculation result is output to the display unit (monitor) 18 (S003). In this manner, it is possible to measure the concentration of hydrogen sulfide.

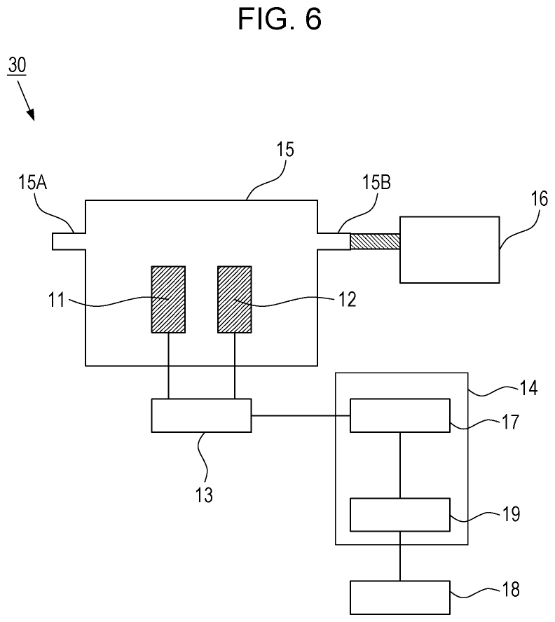

The gas evaluation apparatus in the technology in the disclosure and a halitosis evaluation method using the gas evaluation apparatus will be described with the drawings. A gas evaluation apparatus 30 illustrated in FIG. 6 includes a first gas sensor device 11, a second gas sensor device 12, a resistance measurement unit 13, a calculation unit 14, a measurement chamber 15, a suction device 16, and a display unit 18.

In the gas evaluation apparatus 30, the first gas sensor device 11 and the second gas sensor device 12 are disposed in the measurement chamber 15 including a gas inlet 15A and a gas outlet 15B. The gas outlet 15B is connected to the suction device 16. The first gas sensor device 11 and the second gas sensor device 12 are connected to the resistance measurement unit 13.

The resistance measurement unit 13 includes, for example, a resistance measuring instrument that measures the resistance change of the first gas sensor device 11 and a resistance measuring instrument that measures the resistance change of the second gas sensor device 12. The resistance measurement unit 13 is connected to the calculation unit 14.

The calculation unit 14 includes a CPU 17 and a memory 19. The calculation unit 14 is connected to the display unit 18.

In the gas evaluation apparatus illustrated in FIG. 6, while breath exhaled from the mouth is caused to flow from the gas inlet 15A into the measurement chamber 15, the gas in the measurement chamber 15 is sucked by the suction device 16 connected to the gas outlet 15B. In this manner, hydrogen sulfide and mercaptans contained in the breath exhaled from the mouth pass by the surface of the conductor in the first gas sensor device 11 and the surface of the conductor in the second gas sensor device 12 which are disposed in the measurement chamber 15. The resistance change of the first gas sensor device 11 and the resistance change of the second gas sensor device 12 at this time are measured by the resistance measurement unit 13. The calculation unit 14 calculates the concentration of hydrogen sulfide and the concentration of mercaptans based on the resistance changes measured by the resistance measurement unit 13. The calculation unit 14 further evaluates odor based on the concentration of hydrogen sulfide and the concentration of mercaptans which have been calculated. The evaluation result is output to the display unit 18. The display unit 18 is a monitor, for example.

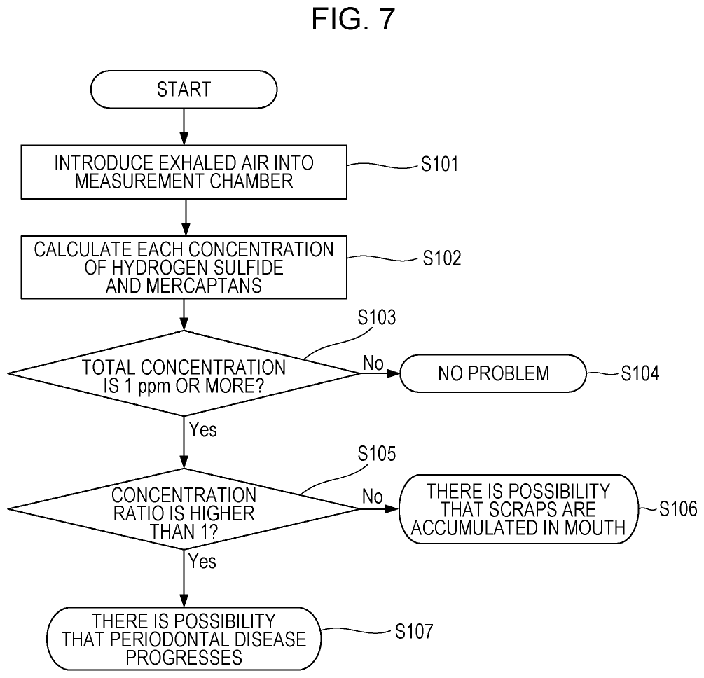

An example of evaluating halitosis using the gas evaluation apparatus illustrated in FIG. 6 will be described with reference to the flowchart in FIG. 7. Firstly, breath (exhalation) exhaled from the mouth is put into the measurement chamber 15 (S101). Then, the concentration of hydrogen sulfide and the concentration of mercaptans in the exhalation are calculated by the first gas sensor device 11, the second gas sensor device 12, the resistance measurement unit 13, and the calculation unit 14 (S102). Then, the calculation unit 14 determines whether or not the sum value of the concentration of hydrogen sulfide and the concentration of mercaptans is equal to or more than a threshold value (for example, 1 ppm) (S103). In a case where the sum value of the concentration of hydrogen sulfide and the concentration of mercaptans is less than the threshold value (for example, 1 ppm), the calculation unit outputs an evaluation result indicating that there is no problem, to the display unit (monitor) 18 (S104).

In a case where the sum value of the concentration of hydrogen sulfide and the concentration of mercaptans is equal to or more than the threshold value, the calculation unit further evaluates the ratio of the concentration of mercaptans to the concentration of hydrogen sulfide (S105). In a case where the ratio of the concentration of mercaptans to the concentration of hydrogen sulfide is equal to or less than 1, the evaluation result indicating a possibility that scraps have accumulated in the mouth is output to the display unit (monitor) 18 (S106). In a case where the ratio of the concentration of mercaptans to the concentration of hydrogen sulfide is more than 1, the evaluation result indicating a possibility that periodontal disease is in progress is output to the display unit (monitor) 18 (S107). In this manner, it is possible to evaluate halitosis.

### EXAMPLES

Examples of the technology in the disclosure will be described below. However, the technology in the disclosure is not limited to the following examples.

### Example 1

Two gold electrodes having a width of 5 mm, a length of 6 mm, and a film thickness of 60 nm were formed on a silicon wafer at a distance set to 1 mm, by using vacuum deposition. The silicon wafer had a thermal oxide film (thickness of 100 nm) and had a width of 15 mm, a length of 15 mm, and a thickness of 0.6 mm. A gold film having a width of 80 μm and a thickness of 6 nm was produced using vacuum deposition, so as to connect the pair of gold electrodes, and thereby a structural body was obtained. Annealing was performed at 200° C. for 10 minutes in an atmosphere to dean the surface of the gold film. Then, the structural body was immersed in a methanol solution containing each of ethylene dioxydiethanethiol and n-hexadecanethiol at 1 volume %, for 60 seconds. Then, the structural body was washed with pure methanol. In this manner, a gas sensor device in which the surface of the gold film was coated with an organic compound having a mercapto group was produced.

The organic compound which had a mercapto group and adhered to the surfaces of the pair of gold electrodes was removed by rubbing the surface of each of the gold electrodes with a cotton swab.

The gas sensor device was installed in an air stream. At room temperature, a gas source was switched between (1) dean air, (2) an air containing hydrogen sulfide at a concentration of 0.2 ppm, and (3) an air containing methyl mercaptan at a concentration of 0.2 ppm, and thereby responses of the device to the gases were evaluated. The temperature of the air (gas) used here was about 24° C., and relative humidity was about 40%. Specifically, evaluation was performed with a method as follows.

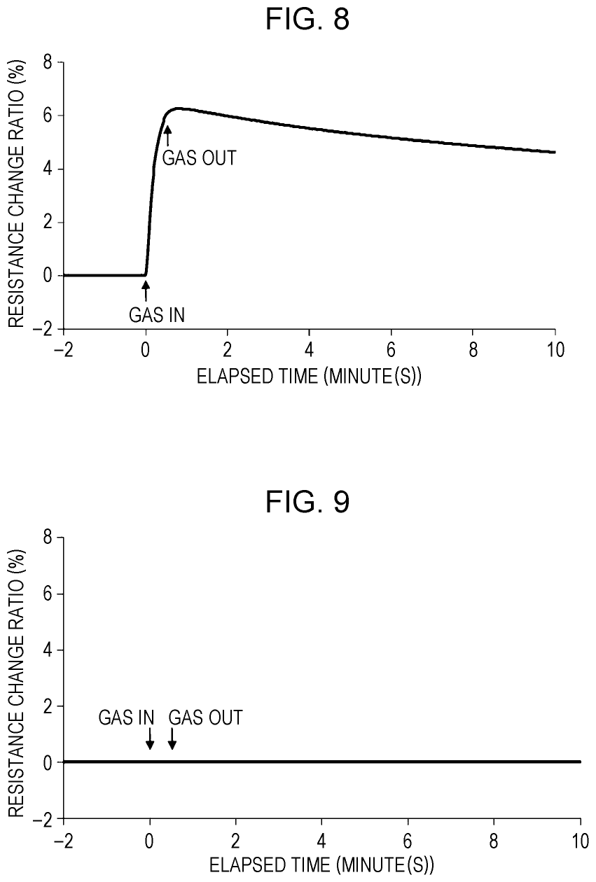

The gas sensor device was installed in an evaluation chamber having an inside coated with fluorinated resin and an inner volume of 150 mL. A response of electrical resistance of the gas sensor device to hydrogen sulfide in a case where an evaluation gas was put into at a flow rate of 1.8 L per minute is illustrated in FIG. 8. A response of electrical resistance of the gas sensor device to methyl mercaptan in a case where the evaluation gas was put into at a flow rate of 1.8 L per minute is illustrated in FIG. 9. Very high gas species selectivity for hydrogen sulfide is exhibited from FIGS. 8 and 9. The resistance value of this device did not exhibit a noticeable change for relative humidity in clean air, which varies from 20% to 65%.

In a case where a methanol solution containing n-hexadecanethiol at 1 volume % was used as a solution used for combining the organic compound having a mercapto group to the surface of the gold film, a similar result was also obtained.

In a case where a methanol solution respectively containing ethylene dioxydiethanethiol and n-hexadecanethiol at 0.7 volume % and 1.3 volume % was used as the solution used for combining the organic compound having a mercapto group to the surface of the gold film, a similar result was also obtained.

### Comparative Example 1

Two gold electrodes having a width of 5 mm, a length of 6 mm, and a film thickness of 60 nm were formed on a silicon wafer at a distance set to 1 mm, by using vacuum deposition. The silicon wafer had a thermal oxide film (thickness of 100 nm) and had a width of 15 mm, a length of 15 mm, and a thickness of 0.6 mm. A gold film having a width of 80 μm and a thickness of 6 nm was produced using vacuum deposition, so as to connect the pair of gold electrodes. Then, a gas sensor device was produced by a method of performing annealing at 200° C. for 10 minutes in an atmosphere.

The gas sensor device was installed in an air stream. At room temperature, a gas source was switched between (1) dean air, (2) an air containing hydrogen sulfide at a concentration of 0.2 ppm, and (3) an air containing methyl mercaptan at a concentration of 0.2 ppm, and thereby responses of the device to the gases were evaluated. The temperature of the air (gas) used here was about 24° C., and relative humidity was about 40%.

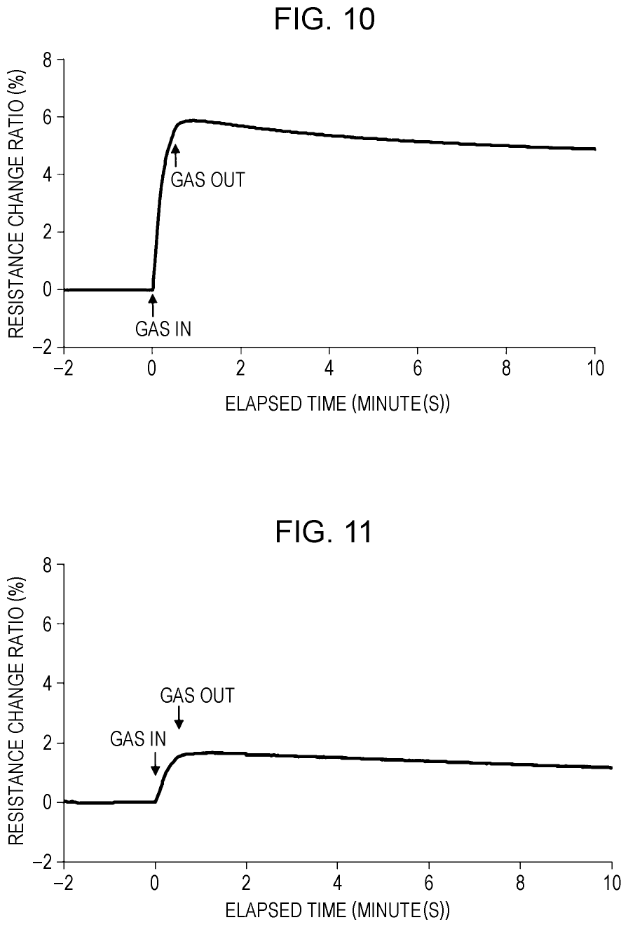

FIG. 10 illustrates a response of electrical resistance of the gas sensor device to hydrogen sulfide in the same condition as that in the method described Example 1. FIG. 11 illustrates a response of electrical resistance of the gas sensor device to methyl mercaptan in the same condition as that in the method described Example 1. Although the intensity of the response is different, it is represented that this device reacts with both hydrogen sulfide and methyl mercaptan. The resistance value of this device did not exhibit a noticeable change for relative humidity in clean air, which varies from 20% to 65%.

### Example 2

The gas sensor device (referred to as “a first gas sensor device” below) produced in Example 1 and the gas sensor device (referred to as “a second gas sensor device” below) produced in Comparative Example 1 were installed one by one in an evaluation chamber. A circuit for measuring the resistance of each of the devices was configured. The evaluation chamber has an inside coated with fluorinated resin and has an inner volume of 40 mL.

A tube for putting an air and a tube for discharging the air were provided in the chamber. The tubes are made of fluorinated resin and have a length of 20 cm and an inner diameter of 6 mm. A suction pump was connected to the tip of the tube for discharging the air.

An air drawn from the mouth was set to pass through the chamber at a flow rate of 500 mL per minute, by using the suction pump. When the air in the mouth was evaluated, the air in the mouth was sucked for 10 seconds, and a rate of the resistance change of each device, which occurred for 5 seconds after 5 seconds elapsed from the start of the suction was set as an analysis target. An analysis method is as follows.

Measurement was performed using several types of gases in which the concentration of hydrogen sulfide contained in an air was 0.2 ppm to 4 ppm, in advance. A calibration curve for a response of the first gas sensor device in terms of a response to pure hydrogen sulfide was created.

Then, a calibration curve of a response to a gas mixture in which a mixing ratio of hydrogen sulfide and methyl mercaptan was 1:1 was created from a response of the second gas sensor device in a case using the gas mixture including hydrogen sulfide and methyl mercaptan (concentration ratio of 1:1) at the same concentration as that in the gas used for creating the calibration curve.

When the air in the mouth was evaluated, the response of the first gas sensor device to the air in the mouth is calculated in terms of the concentration of hydrogen sulfide by referring to the calibration curve of the first gas sensor device. The response of the second gas sensor device is calculated in terms of the concentration of a gas in which the mixing ratio of hydrogen sulfide and methyl mercaptan is 1:1, by referring to the calibration curve of the device. The approximate concentration of mercaptans corresponding to the concentration of methyl mercaptan is obtained by subtracting the concentration of hydrogen sulfide obtained from the first gas sensor device, from the obtained gas concentration.

In the gas evaluation apparatus produced in the above manner, for example, the exhalation may be evaluated in accordance with the flowchart in FIG. 7. A function of determining that “halitosis is strong” in a case where the sum (sum value) of the concentration of hydrogen sulfide and the approximate concentration of mercaptans corresponding to methyl mercaptan is equal to or more than 1 ppm and displaying a message that halitosis is strong may be provided. A configuration in which it is determined that “there is a possibility that periodontal disease is in progress”, in a case where the approximate concentration of mercaptans corresponding to methyl mercaptan is more than twice the concentration of hydrogen sulfide in a case where it is determined that “halitosis is strong”, and a display for warning periodontal disease is performed may be made. A configuration in which it is determined that “there is a possibility that the tongue has largely furred”, in a case where the approximate concentration of mercaptans corresponding to methyl mercaptan is equal to or less than twice the concentration of hydrogen sulfide in a case where it is determined that “halitosis is strong”, and a display for urging cleaning of the mouth is performed may be made.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A gas sensor device comprising:
a pair of electrodes;
a conductor that electrically connects the pair of electrodes; and
at least one of a thiolate anion and an organic compound having a mercapto group, having been disposed on a surface of the conductor.

2. The gas sensor device according to claim 1,
wherein a material of the conductor is gold or platinum-group metal.

3. The gas sensor device according to claim 1,
wherein a material of the conductor is gold, and
at least the organic compound is disposed in an area which is on the surface of the conductor and in which a crystal plane of the gold is (111).

4. A manufacturing method of a gas sensor device, the method comprising:
forming a structural body including a pair of electrodes and a conductor that electrically connects the pair of electrodes on an insulating substrate; and
bringing the conductor into contact with a solution containing an organic compound having a mercapto group.

5. A gas evaluation apparatus comprising:
a first gas sensor device including
a pair of first electrodes,
a first conductor that electrically connects the pair of first electrodes, and
at least one of a thiolate anion and an organic compound having a mercapto group, having been disposed on a surface of the first conductor; and
a resistance measurement circuit that measures resistance of the first gas sensor device.

6. The gas evaluation apparatus according to claim 5, further comprising:
a second gas sensor device including
a pair of second electrodes, and
a second conductor that electrically connects the pair of second electrodes,
wherein neither the mercapto group nor the thiolate anion is disposed on the second conductor,
wherein the resistance measurement circuit further measures resistance of the second gas sensor device.

7. The gas evaluation apparatus according to claim 6, further comprising:
a calculation circuit that calculates a concentration of each of hydrogen sulfide and mercaptans based on the resistances measured by the resistance measurement circuit.

8. The gas evaluation apparatus according to claim 7, further comprising:
a display connected to the calculation circuit,
wherein the calculation circuit outputs an evaluation result depending on the concentrations of the hydrogen sulfide and the mercaptans.

9. The gas evaluation apparatus according to claim 5, further comprising:
a measurement chamber that accommodates the first gas sensor device.

10. The gas evaluation apparatus according to claim 5,
wherein a material of the first conductor is gold or platinum-group metal.

11. The gas evaluation apparatus according to claim 6,
wherein a material of the second conductor is gold or platinum-group metal.

12. The gas evaluation apparatus according to claim 6, further comprising:
a measurement chamber that accommodates the first gas sensor device and the second gas sensor device.

13. The gas evaluation apparatus according to claim 7, wherein
the calculation circuit further calculates a ratio of the concentration of the mercaptans to the concentration of the hydrogen sulfide when a sum of the concentration of the hydrogen sulfide and the concentration of the mercaptans are greater than a threshold.

14. The gas evaluation apparatus according to claim 13, further comprising:
a chamber housing the first gas sensor device and second house sensor device and including gas inlet through which a user breathes into a chamber and a gas outlet through which the user's breath exits the chamber,
the calculated ratio being compared to a predetermined value to diagnose a characteristic of the user.

15. The gas evaluation apparatus according to claim 14, wherein the characteristic of the user relates to halitosis.
